(19) United States
(12) Patent Application Publication
(10) Pub. No.: US 2011/0195128 A1
(43) Pub. Date: Aug. 11, 2011

(54) TREATING OR PREVENTING GOUT

(76) Inventors: Kay Patricia Palmano, Palmerston North (NZ); Alastair Kenneth Hugh MacGibbon, Palmerston North (NZ); Nicole Jane Moore Dalbeth, Auckland (NZ)

(21) Appl. No.: 13/123,482

(22) PCT Filed: Oct. 9, 2009
(86) PCT No.: PCT/NZ09/00215

§ 371 Date: Apr. 29, 2011

(30) Foreign Application Priority Data
Oct. 9, 2008 (NZ) 571856

(51) Int. Cl. A61K 35/20; A61K 31/7004; A61K 38/00; A61K 38/16; A61P 43/00

(52) U.S. Cl. 424/535; 514/25; 514/1.1; 514/21.3

(57) ABSTRACT

Use of glycomacropeptide to treat or prevent gout, use of one or more phospholipids and one or more gangliosides to treat or prevent gout, or use of glycomacropeptide and one or more phospholipids and one or more gangliosides to treat or prevent gout.

### FIELD OF THE INVENTION

The present invention relates to the use of glycomacropeptide to treat or prevent gout, use of one or more phospholipids and one or more gangliosides to treat or prevent gout or use of a combination of glycomacropeptide, one or more phospholipids and one or more gangliosides to treat or prevent gout.

### BACKGROUND

### Gout and Hyperuricaemia

Gout, also called metabolic arthritis, is the most common form of inflammatory arthritis affecting men. Gout is more prevalent in men between the ages of 50 and 60 and also has a higher propensity in some races. The occurrence of gout is high among New Zealand Maori and Pacific Islanders (Roddy et al 2007).

Purine metabolism results in uric acid being formed. In a healthy individual, uric acid is normally excreted in the urine. In some people however, the balance between uric acid production and excretion is impaired which results in elevated levels of uric acid in the bloodstream (hyperuricaemia). Some hyperuricaemia sufferers will develop gout (Campion et al 1987).

### Pathology of Gout

Gout is uniquely characterised by the deposition of monosodium urate (MSU) crystals on the articular cartilage of joints, tendons and surrounding tissues (Martinon and Glimcher 2006). These MSU crystals provoke an inflammatory response which causes sudden and excruciating pain in the joints in which the MSU crystals are deposited. This inflammatory response is characterised by two phases and involves the activation of the NALP-3 inflammasome.

The first of the inflammatory phases is believed to involve the internalisation of the MSU crystals by phagocytic cells, such as monocytes. Once internalised, the uric acid crystals activate the NALP-3 inflammasome resulting in the processing and maturation of pro-IL-1β. Mature IL-1β will then be secreted from the phagocytic cell and initiate phase 2 of the gout inflammatory response (Martinon and Glimcher 2006).

It is reported that phase 2 of the gout inflammatory response corresponds to inflammation through an IL-1R/MyD88 dependent pathway in non-bone marrow derived cells resulting in the production of chemokines, including IL-8 and MIP-2, and inflammatory mediators that cause an influx of neutrophils to the joints affected by gout (Martinon and Glimcher 2006).

### Treatment of Gout

Non-steroidal anti-inflammatory drugs (NSAID) are often used in the treatment of gout. In some parts of the world, including the United Kingdom, indometacin is the most popular treatment of gout (Underwood 2006). However, there are no comparative studies showing that indometacin is the most effective NSAID, and the modest amount of published data that is available suggests that there is no difference in the efficacy or tolerability between any of the NSAIDs (Drug therapeutics bulletin 2004). It is also well documented that NSAIDs have a number of gastrointestinal and cardiovascular risks associated with them (Underwood 2006).

Colchicine has also been used to treat gout in patients that cannot tolerate NSAIDs. One published study showed that within 48 hours a 50% reduction in pain was achieved in joints treated with colchicine. However, all patients taking colchicine developed diarrhoea and/or vomiting within 24 hours of taking this medication (Drug therapeutics bulletin 2004).

For patients who suffer gout attacks, the best treatment is a lifestyle plan that will prevent acute gout attacks from occurring. Reducing body weight and changing diet can alter serum uric acid levels, but some patients find this difficult to achieve. Additionally, a recently published study reports support for the traditional advice of reducing alcohol and purine-rich food consumption. (Drug therapeutics bulletin 2004).

Along with a change in lifestyle, prophylactic drugs are also sometimes prescribed for the prevention of gout attacks. Allopurinol is a xanthine oxidase inhibitor that reduces serum concentrations of uric acid by blocking its synthesis. Sulfinpyrazone is a uricosuric agent that decreases serum concentrations of uric acid by increasing renal excretion. However, there does not appear to be any published data showing that either of these drugs decreases the reoccurrence of gout (Drug therapeutics bulletin 2004).

Therefore, a need exists for alternative therapies to treat or prevent gout. It would therefore be desirable to provide an alternative therapy to treat or prevent gout or to at least provide the public with a useful choice.

### SUMMARY OF THE INVENTION

Accordingly, in a first aspect the present invention relates to use of one or more agents in the manufacture of a composition for treating or preventing gout, wherein the one or more agents is selected from:

(a) glycomacropeptide,
(b) an isolated peptide consisting essentially of or consisting of SEQ ID NO. 1 or 2 or a functional fragment thereof,
(c) a functional variant of (b) having at least 80% sequence identity to SEQ ID NO. 1 or 2 or a functional fragment thereof,
(d) a peptide of about 64 to about 100 amino acids in length, wherein the peptide comprises SEQ ID NO. 1 or 2,
(e) a functional variant of (d), wherein the peptide comprises an amino acid sequence having at least 80% sequence identity to SEQ ID NO. 1 or 2,
(f) a casein hydrolysate comprising one or more peptides consisting of SEQ ID NO. 1 or 2, wherein the hydrolysate comprises about 51 to about 100% by weight of the one or more peptides,
(g) sweet whey, and
(h) any combination of any two or more of (a) to (g).

In a second aspect the present invention relates to use of one or more agents in the manufacture of a composition for treating or preventing gout, wherein the one or more agents is selected from:

(a) about 0.1 to about 99.9% by weight phospholipid and about 0.1 to about 99.9% by weight ganglioside,
(b) a milk fat extract comprising about 0.1 to about 99.9% by weight phospholipid and about 0.1 to about 99.9% by weight ganglioside,
(c) a milk fat extract comprising about 1 to about 90% lipid wherein the lipid comprises about 0.1 to about 99.9% by weight phospholipid and about 0.1 to about 99.9% by weight ganglioside, and
(d) any combination of any two or more of (a) to (c).

In a third aspect the present invention relates to use of

(i) one or more agents as defined in the first aspect, and
(ii) one or more agents as defined in the second aspect,
in the manufacture of a composition for treating or preventing gout.

In a fourth aspect the present invention relates to one or more agents for treating or preventing gout, wherein the one or more agents is selected from:

(a) glycomacropeptide,
(b) an isolated peptide consisting essentially of or consisting of SEQ ID NO. 1 or 2 or a functional fragment thereof,
(c) a functional variant of (b) having at least 80% sequence identity to SEQ ID NO. 1 or 2 or a functional fragment thereof,
(d) a peptide of about 64 to about 100 amino acids in length, wherein the peptide comprises SEQ ID NO. 1 or 2,
(e) a functional variant of (d), wherein the peptide comprises an amino acid sequence having at least 80% sequence identity to SEQ ID NO. 1 or 2,
(f) a casein hydrolysate comprising one or more peptides consisting of SEQ ID NO. 1 or 2, wherein the hydrolysate comprises about 51 to about 100% by weight of the one or more peptides,
(g) sweet whey, and
(h) any combination of any two or more of (a) to (g).

In a fifth aspect the present invention relates to one or more agents for treating or preventing gout, wherein the one or more agents is selected from:

(a) about 0.1 to about 99.9% by weight phospholipid and about 0.1 to about 99.9% by weight ganglioside,
(b) a milk fat extract comprising about 0.1 to about 99.9% by weight phospholipid and about 0.1 to about 99.9% by weight ganglioside,
(c) a milk fat extract comprising about 1 to about 90% lipid wherein the lipid comprises about 0.1 to about 99.9% by weight phospholipid and about 0.1 to about 99.9% by weight ganglioside, and
(d) any combination of any two or more of (a) to (c).

In a sixth aspect the present invention relates to one or more agents for treating or preventing gout, wherein the one or more agents is selected from:

(i) one or more agents as defined in the fourth aspect, and
(ii) one or more agents as defined in the fifth aspect.

In a seventh aspect the present invention relates to a method for treating or preventing gout comprising administering an effective amount of one or more agents to a subject in need thereof, wherein the one or more agents is selected from:

(a) glycomacropeptide,
(b) an isolated peptide consisting essentially of or consisting of SEQ ID NO. 1 or 2 or a functional fragment thereof,
(c) a functional variant of (b) having at least 80% sequence identity to SEQ ID NO. 1 or 2 or a functional fragment thereof,
(d) a peptide of about 64 to about 100 amino acids in length, wherein the peptide comprises SEQ ID NO. 1 or 2,
(e) a functional variant of (d), wherein the peptide comprises an amino acid sequence having at least 80% sequence identity to SEQ ID NO. 1 or 2,
(f) a casein hydrolysate comprising one or more peptides consisting of SEQ ID NO. 1 or 2, wherein the hydrosylate comprises about 51 to about 100% by weight of the one or more peptides,
(g) sweet whey, and
(h) any combination of any two or more of (a) to (g).

In an eighth aspect the present invention relates to a method for treating or preventing gout comprising administering an effective amount of one or more agents to a subject in need thereof, wherein the one or more agents is selected from:

(a) about 0.1 to about 99.9% by weight phospholipid and about 0.1 to about 99.9% by weight ganglioside,
(b) a milk fat extract comprising about 0.1 to about 99.9% by weight phospholipid and about 0.1 to about 99.9% by weight ganglioside,
(c) a milk fat extract comprising about 1 to about 90% lipid wherein the lipid comprises about 0.1 to about 99.9% by weight phospholipid and about 0.1 to about 99.9% by weight ganglioside, and
(d) any combination of any two or more of (a) to (c).

In a ninth aspect the present invention relates to a method of treating or preventing gout comprising administering an effective amount of one or more agents as defined in the seventh aspect and one or more agents as defined in the eighth aspect to a subject in need thereof.

In a tenth aspect the present invention relates to a composition comprising one or more agents as defined in the first aspect and one or more agents as defined in the second aspect.

The following embodiments may relate to any of the above aspects.

In one embodiment a composition or product useful herein comprises at least about 51% of an agent as defined in the first or fourth aspect. In yet another embodiment a method of the invention comprises administration of a composition comprising at least about 51% of an agent as defined in the seventh aspect.

In one embodiment a composition or product useful herein is formulated for separate, simultaneous or sequential administration of the one or more agents. For example the composition may be formulated for separate, simultaneous or sequential administration of the ganglioside and the phospholipid. In other embodiments the composition may be formulated for separate, simultaneous or sequential administration of the glycomacropeptide, the ganglioside and the phospholipid. In a method of the invention the agents may also be administered separately, simultaneously or sequentially.

In one embodiment a composition, product or method useful herein comprises or comprises administration of at least about 0, 0.1, 0.2, 0.3, 0.4, 0.5, 0.6, 0.7, 0.8, 0.9, 1.0, 1 5, 2, 2.5, 3, 3.5, 4, 4.5, 5, 5.5, 6, 6.5, 7, 7.5 8, 8.5, 9, 9.5, 10, 15, 20, 25, 30, 35, 40, 45, 50, 55, 60, 65, 70, 75, 80, 85, 90, 95, 99, 99.5 or 99.9% by weight total lipid, and useful ranges may be selected between any of these values (for example, about 0.1 to about 95%, about 5 to about 95%, about 10 to about 95%, about 15 to about 95%, about 20 to about 95%, about 25 to about 95%, about 30 to about 95%, about 35 to about 95%, about 40 to about 95%, about 45 to about 95%, about 50 to about 95%, about 5 to about 99%, about 10 to about 99%, about 15 to about 99%, about 20 to about 99%, about 25 to about 99%, about 30 to about 99%, about 35 to about 99%, about 40 to about 99%, about 45 to about 99%, about 50 to about 99%, about 5 to about 70%, about 10 to about 70%, about 15 to about 70%, about 20 to about 70%, about 25 to about 70%, about 30 to about 70%, about 35 to about 70%, about 40 to about 70%, about 45 to about 70%, about 50 to about 70%, about 0 to about 1%, about 0 to about 2%, about 0 to about 3%, about 0 to about 4, about 0 to about 5, 0 to about 6%, 0 to about 7%, 0 to about 8%, 0 to about 9%, about 0 to about 10%, about 0.1 to about 1%, about 0.1 to about 2%, about 0.1 to about 3%, about 0.1 to about 4, about 0.1 to about 5, 0.1 to about 6%, 0.1 to about 7%, 0.1 to about 8%, 0.1 to about 9% and about 0.1 to about 10% by weight total lipid).

In one embodiment a composition, product or method useful herein comprises or comprises administration of at least about 0.1, 0.2, 0.3, 0.4, 0.5, 0.6, 0.7, 0.8, 0.9, 1.0, 1 5, 2, 2.5, 3, 3.5, 4, 4.5, 5, 5.5, 6, 6.5, 7, 7.5 8, 8.5, 9, 9.5, 10, 15, 20, 25, 30, 35, 40, 45, 50, 55, 60, 65, 70, 75, 80, 85, 90, 95, 99, 99.5 or 99.9% by weight phospholipid, and useful ranges may be selected between any of these values (for example, about 5 to about 95%, about 10 to about 95%, about 15 to about 95%, about 20 to about 95%, about 25 to about 95%, about 30 to about 95%, about 35 to about 95%, about 40 to about 95%, about 45 to about 95%, about 50 to about 95%, about 5 to about 99%, about 10 to about 99%, about 15 to about 99%, about 20 to about 99%, about 25 to about 99%, about 30 to about 99%, about 35 to about 99%, about 40 to about 99%, about 45 to about 99%, about 50 to about 99%, about 5 to about 70%, about 10 to about 70%, about 15 to about 70%, about 20 to about 70%, about 25 to about 70%, about 30 to about 70%, about 35 to about 70%, about 40 to about 70%, about 45 to about 70%, and about 50 to about 70% by weight phospholipid). The phospholipid may comprise one or more phospholipids selected from phosphatidylcholine, phosphatidylethanolamine, sphingomyelin, phosphatidylserine, and phosphatidylinositol, or any combination of any two or more thereof. In some embodiments the phospholipid may comprise two or more, three or more, four or more or all of phosphatidylcholine, phosphatidylethanolamine, sphingomyelin, phosphatidylserine, and phosphatidylinositol.

In one embodiment a composition, product or method useful herein comprises or comprises administration of at least about 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of phosphatidylcholine, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% by weight of phosphatidylcholine). In an alternative embodiment, the composition may further comprise one or more, two or more, or three or more additional phospholipids selected from phosphatidylethanolamine, sphingomyelin, phosphatidylserine, and phosphatidylinositol. Suitable ranges of these additional phospholipids may also be selected between any of the values defined above.

In one embodiment a composition, product or method useful herein comprises or comprises administration of at least about 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of phosphatidylethanolamine, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% by weight of phosphatidylethanolamine). In an alternative embodiment, the composition may further comprise one or more, two or more, or three or more additional phospholipids selected from phosphatidylcholine, sphingomyelin, phosphatidylserine, and phosphatidylinositol. Suitable ranges of these additional phospholipids may also be selected between any of the values defined above.

In one embodiment a composition, product or method useful herein comprises or comprises administration of at least about 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of sphingomyelin, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% by weight of sphingomyelin). In an alternative embodiment, the composition may further comprise one or more, two or more, or three or more additional phospholipids selected from phosphatidylcholine, phosphatidylethanolamine, phosphatidylserine, and phosphatidylinositol. Suitable ranges of these additional phospholipids may also be selected between any of the values defined above.

In one embodiment a composition, product or method useful herein comprises or comprises administration of at least about 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of phosphatidylserine, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% by weight of phosphatidylserine). In an alternative embodiment, the composition may further comprise one or more, two or more, or three or more additional phospholipids selected from phosphatidylcholine, phosphatidylethanolamine, sphingomyelin, and phosphatidylinositol. Suitable ranges of these additional phospholipids may also be selected between any of the values defined above.

In one embodiment a composition, product or method useful herein comprises or comprises administration of at least about 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of phosphatidylinositol, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% by weight of phosphatidylinositol). In an alternative embodiment, the composition may further comprise one or more, two or more, or three or more additional phospholipids selected from phosphatidylcholine, phosphatidylethanolamine, sphingomyelin, and phosphatidylserine. Suitable ranges of these additional phospholipids may also be selected between any of the values defined above.

In one embodiment a composition, product or method useful herein comprises or comprises administration of at least about 15% to about 99% by weight total lipid, about 1% to about 80% by weight total phospholipid. In one embodiment the composition, product or method useful herein comprises or comprises administration of about 1% to about 25% by weight phosphatidylcholine, about 0.1% to about 15% by weight phosphatidylinositol, about 0.1% to about 15% by weight phosphatidylserine, about 1% to about 30% by weight phosphatidylethanolamine, and about 0.5% to about 25% by weight sphingomyelin.

In one embodiment a composition, product or method useful herein comprises or comprises administration of about 1% to about 60% by weight lactose, about 1% to about 15% by weight lactose or about 50% to about 65% by weight lactose. In alternative embodiments, the composition, product or method useful herein comprises or comprises administration of about 20% to about 40% by weight total lipid, about 5% to about 25% by weight total phospholipid, and amounts of one or more phospholipids as described above. In other alternative embodiments, the composition, product or method useful herein comprises or comprises administration of about 70% to about 99% by weight total lipid, about 25% to about 80% by weight total phospholipid, and amounts of one or more phospholipids as described above. In still further alternative embodiments, the composition, product or method useful herein comprises or comprises administration of about 0.1% to about 5%, about 0.1% to about 2.5%, or about 3% to about 5% by weight of one or more gangliosides, preferably independently selected from GD3 and GM3.

In one embodiment the composition, product or method useful herein comprises or comprises administration of at least about 15 to 40% total lipid, about 1% to about 6% phosphatidylcholine, about 1% to about 6% phosphatidylinositol, about 1% to about 6% phosphatidylserine, about 1% to about 6% phosphatidylethanolamine, and about 1% to about 3% sphingomyelin. In a preferred embodiment, the composition, product or method useful herein comprises or comprises administration of at least about 3% to about 6% myristic acid (14:0), at least about 12% to about 20% palmitic acid (16:0), at least about 0.5% to about 3% palmitoleic acid (16:1), at least about 0.1% to about 1.5% margaric acid (17:0), at least about 13% to about 20% stearic acid (18:0), at least about 28% to about 35% oleic acid (18:1), at least about 3% to about 5% linoleic acid (18:2) and at least about 0.5% to about 2.5% linolenic (18:3). In one embodiment, the composition, product or method useful herein comprises or comprises administration of about 1% to about 2.5% ganglioside GD3, about 0.1% to about 1% ganglioside GM3, or both.

In one embodiment the composition, product or method useful herein comprises or comprises administration of one or more phosphatidylethanolamines, one or more phosphatidylinositols, one or more phosphatidylserines, one or more phosphatidylcholines, one or more sphingolipids (including one or more sphingomyelins, one or more dihydrosphingomyelins, one or more ceramides, one or more cerebrosides, or one or more gangliosides, or any combination of any two or more thereof), one or more lysophospholipids (phospholipids with one fatty acid lost), or any combination of any two or more thereof.

In one embodiment the composition, product or method useful herein comprises or comprises administration of at least about 0.1, 0.2, 0.3, 0.4, 0.5, 0.6, 0.7, 0.8, 0.9, 1.0, 1 5, 2, 2.5, 3, 3.5, 4, 4.5, 5, 5.5, 6, 6.5, 7, 7.5 8, 8.5, 9, 9.5, 10, 15, 20, 25, 30, 35, 40, 45, 50, 55, 60, 65, 70, 75, 80, 85, 90, 95, 99.5 or 99.9% by weight ganglioside, and useful ranges may be selected between any of these values (for example, about 0.1 to about 1.0, about 0.1 to about 5.0, about 0.1 to about 10, about 0.1 to about 20, about 0.1 to about 30, about 0.1 to about 40, about 0.1 to about 50, about 0.1 to about 60, about 0.1 to about 70, about 0.1 to about 80, about 0.1 to about 90, about 0.1 to about 99, about 5 to about 95%, about 10 to about 95%, about 15 to about 95%, about 20 to about 95%, about 25 to about 95%, about 30 to about 95%, about 35 to about 95%, about 40 to about 95%, about 45 to about 95%, about 50 to about 95%, about 10 to about 70%, about 15 to about 70%, about 20 to about 70%, about 25 to about 70%, about 30 to about 70%, about 35 to about 70%, about 40 to about 70%, about 45 to about 70%, and about 50 to about 70% by weight ganglioside). In one embodiment the composition, product or method useful herein comprises or comprises administration of GD3 or GM3 or a combination thereof.

In one embodiment the composition, product or method useful herein comprises or comprises administration of about 0.1% to about 30% by weight of one or more gangliosides. More preferably, the composition, product or method useful herein comprises or comprises administration of about 0.1% to about 5% by weight of one or more gangliosides.

In one embodiment the composition, product or method useful herein comprises or comprises administration of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of ganglioside GD3, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% by weight of GD3. In an alternative embodiment, the composition may further comprise one or more additional gangliosides, for example GM3.

In one embodiment the composition, product or method useful herein comprises or comprises administration of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 19, 20, 21, 22, 23, 24, 25, 26, 27, 28, 29 or 30% by weight of ganglioside GM3, and useful ranges may be selected between any of these values (for example, about 0.1 to about 30%, about 0.5 to about 30%, about 1 to about 30%, about 2 to about 30%, about 3 to about 30%, about 4 to about 30%, about 5 to about 30%, about 10 to about 30%, about 15 to about 30%, about 20 to about 30%, about 0.1 to about 5%, about 0.5 to about 5%, about 1 to about 5%, about 2 to about 5%, about 3 to about 5%, about 0.1 to about 10%, about 0.5 to about 10%, about 1 to about 10%, about 2 to about 10%, about 3 to about 10%, about 4 to about 10%, about 5 to about 10%, about 6 to about 10%, about 0.1 to about 20%, about 0.5 to about 20%, about 1 to about 20%, about 2 to about 20%, about 3 to about 20%, about 4 to about 20%, about 5 to about 20%, about 10 to about 20%, about 15 to about 20% by weight of GM3. In an alternative embodiment, the composition may further comprise one or more additional gangliosides, for example GD3.

In one embodiment a method useful herein comprises administration of one or more agents as defined herein in a composition or product.

In one embodiment a composition or product useful herein is a food, confectionary, milk, milk product, milk powder, reconstituted milk, cultured milk, yoghurt, drinking yoghurt, set yoghurt, drink, dairy drink, milk drink, food additive, drink additive, dietary supplement, nutritional product, medical food, nutraceutical or pharmaceutical. In one embodiment the composition or product comprises less than about 4%, 3%, 2% or 1% by weight fat. For example, in one embodiment a composition or product useful herein comprises less than 4% lipid by weight, preferably less than about 3.5, 3, 2.5, 2, 1.5, 1, or 0.5% lipid by weight. For example, a composition or product useful herein comprises about 0% to about 4%, about 0% to about 3.5%, about 0% to about 3%, about 0% to about 2.5%, about 0% to about 2%, about 0% to about 1.5%, about 0% to about 1%, about 0% to about 0.5% lipid by weight.

These compositions or products may include any edible consumer product which is able to carry protein or lipid or both. Examples of suitable edible consumer products include aqueous products, baked goods, confectionary products including chocolate, gels, ice creams, reconstituted fruit products, snack bars, food bars, muesli bars, spreads, sauces, dips, dairy products including yoghurts and cheeses, drinks including dairy and non-dairy based drinks, milk, milk powders, sports supplements including dairy and non-dairy based sports supplements, fruit juice, food additives such as protein sprinkles and dietary supplement products including daily supplement tablets. Suitable nutraceutical compositions useful herein may be provided in similar forms.

In one embodiment a composition or product useful herein is a dairy product. In one embodiment the dairy product useful herein is a milk, milk product, milk powder, reconstituted milk, cultured milk, yoghurt, drinking yoghurt, set yoghurt, dairy drink, or milk drink. In one embodiment the dairy product useful herein comprises less than about 4%, 3%, 2% or 1% by weight fat. In one embodiment, the composition comprises skim milk or skim milk powder. Preferably, the skim milk comprises less than about 1.5% fat by weight. Preferably the skim milk comprises, consists essentially of, or consists of late season skim milk. Preferably, when skim milk is present, the composition decreases or maintains serum uric acid levels or increases uric acid excretion.

In one embodiment the composition, product or method useful herein comprises or comprises administration of about 1 mg to about 1000 mg per kg body weight, about 1 to about 500 mg per kg body weight, about 1 to about 100 mg per kg bodyweight or about 1 to about 30 mg per kg bodyweight of one or more of the agents as defined in the first aspect. Preferably a composition, product or method useful herein provides these agents in these ranges per day.

In one embodiment the composition, product or method useful herein comprises or comprises administration of about 1 mg to about 1000 mg per kg body weight, about 1 to about 500 mg per kg body weight, about 150 to about 410 mg per kg body weight, about 1 to about 100 mg per kg body weight or about 1 to about 20 mg per kg body weight of one or more of the agents as defined in the second aspect. Preferably a composition, product or method useful herein provides these agents in these ranges per day.

In one embodiment, the composition, product or method useful herein comprises or comprises administration of about 1, 5, 10, 15, 20, 25, 30, 35, 40, 45 or 50% by weight, preferably about 1% to about 50% by weight, of one or more agents, the one or more agents comprising about 0.1% to about 99.9% by weight of one or more agents as defined in (a) to (h) of the first aspect, or about 0.1% to about 99.9% by weight of one or more agents as defined in (a) to (d) of the second aspect, or any combination thereof. In another embodiment, the composition comprises a dairy product comprising about 1% to about 50% by weight of one or more agents, the one or more agents comprising about 0.1% to about 99.9% by weight of one or more agents as defined in (a) to (h) of the first aspect, or about 0.1% to about 99.9% by weight of one or more agents as defined in (a) to (d) of the second aspect, or any combination thereof.

In one embodiment a composition or product useful herein further comprises a pharmaceutically acceptable carrier. In one embodiment the composition or product is in the form of a tablet, a caplet, a pill, a hard or soft capsule or a lozenge. In one embodiment the composition or product is in the form of a cachet, a dispensable powder, granules, a suspension, an elixir, a liquid, or any other form that can be added to food or drink, including for example water, milk or fruit juice. In one embodiment the composition or product further comprises one or more constituents (such as antioxidants) which prevent or reduce degradation of the composition or product during storage or after administration. In one embodiment a method useful herein comprises the administration of one or more agents as defined herein in one of the forms defined above.

In one embodiment a composition, product or method useful herein comprises or comprises administration of at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8, 99.9 or 100% by weight of one or more agents as described above and useful ranges may be selected between any of these foregoing values (for example, from about 0.1 to about 50%, about 0.2 to about 50%, about 0.5 to about 50%, about 1 to about 50%, about 5 to about 50%, about 10 to about 50%, about 15 to about 50%, about 20 to about 50%, about 25 to about 50%, about 30 to about 50%, about 35 to about 50%, about 40 to about 50%, about 45 to about 50%, about 0.1 to about 60%, about 0.2 to about 60%, about 0.5 to about 60%, about 1 to about 60%, about 5 to about 60%, about 10 to about 60%, about 15 to about 60%, about 20 to about 60%, about 25 to about 60%, about 30 to about 60%, about 35 to about 60%, about 40 to about 60%, about 45 to about 60%, about 51 to about 60%, about 0.1 to about 70%, about 0.2 to about 70%, about 0.5 to about 70%, about 1 to about 70%, about 5 to about 70%, about 10 to about 70%, about 15 to about 70%, about 20 to about 70%, about 25 to about 70%, about 30 to about 70%, about 35 to about 70%, about 40 to about 70%, about 45 to about 70%, about 51 to about 70%, about 0.1 to about 80%, about 0.2 to about 80%, about 0.5 to about 80%, about 1 to about 80%, about 5 to about 80%, about 10 to about 80%, about 15 to about 80%, about 20 to about 80%, about 25 to about 80%, about 30 to about 80%, about 35 to about 80%, about 40 to about 80%, about 45 to about 80%, about 51 to about 80%, about 0.1 to about 90%, about 0.2 to about 90%, about 0.5 to about 90%, about 1 to about 90%, about 5 to about 90%, about 10 to about 90%, about 15 to about 90%, about 20 to about 90%, about 25 to about 90%, about 30 to about 90%, about 35 to about 90%, about 40 to about 90%, about 45 to about 90%, about 51 to about 90%, about 0.1 to about 99%, about 0.2 to about 99%, about 0.5 to about 99%, about 1 to about 99%, about 5 to about 99%, about 10 to about 99%, about 15 to about 99%, about 20 to about 99%, about 25 to about 99%, about 30 to about 99%, about 35 to about 99%, about 40 to about 99%, about 45 to about 99%, about 51 to about 99, and about 51 to about 100%). Hydrolysed forms of the agents may be used, where hydrolysis is performed using known methods to a desired degree of hydrolysis.

In one embodiment a composition, product or method useful herein comprises or comprises administration of, at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of one or more agents as described above and useful ranges may be selected between any of these foregoing values (for example, from about 0.01 to about 0.5 grams, about 0.01 to about 1 grams, about 0.01 to about 2 grams, about 0.01 to about 3 grams, about 0.01 to about 4 grams, about 0.01 to about 5 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams). In one embodiment a composition or product useful herein comprises from about 0.01 to about 0.5 grams, about 0.01 to about 1 grams, about 0.01 to about 2 grams, about 0.01 to about 3 grams, about 0.01 to about 4 grams, about 0.01 to about 5 grams, or about 0.01 to about 10 grams of one or more agents of the first aspect. In another embodiment a composition or product useful herein comprises from about 0.01 to about 0.5 grams, about 0.01 to about 1 grams, about 0.01 to about 2 grams, about 0.01 to about 3 grams, about 0.01 to about 4 grams, about 0.01 to about 5 grams, or about 0.01 to about 10 grams of one or more agents of the second aspect.

In one embodiment a composition or product useful herein further comprises, consists essentially of, or consists of about 0.1, 0.5, 1, 5, 10, 15, 20, 25, 30, 35, 40, 45, 50, 55, 60, 65, 70, 75, 80, 85, 90 or 95% by weight of fresh whole milk or a milk derivative and useful ranges may be selected between any of these foregoing values (for example, from about 0.1 to about 50%, about 0.2 to about 50%, about 0.5 to about 50%, about 1 to about 50%, about 5 to about 50%, about 10 to about 50%, about 15 to about 50%, about 20 to about 50%, about 25 to about 50%, about 30 to about 50%, about 35 to about 50%, about 40 to about 50%, and about 45 to about 50%). The milk derivative is preferably selected from recombined, powdered or fresh skim milk, recombined or reconstituted whole or skim milk powder, skim milk concentrate, skim milk retentate, concentrated milk, ultrafiltered milk retentate, milk protein concentrate (MPC), milk protein isolate (MPI), calcium depleted milk protein concentrate (MPC), casein, caseinate, milk fat, cream, butter, ghee, anhydrous milk fat (AMF), buttermilk, butter serum, beta serum, hard milk fat extracts, soft milk fat extracts, sphingolipid extracts, milk fat globule membrane extracts, milk fat globule membrane lipid extracts, phospholipid extracts, complex lipid extracts, colostrum, a colostrum extract, colostrum protein concentrate (CPC), colostrum whey, an immunoglobulin extract from colostrum, whey (including sweet whey, lactic acid whey, mineral acid whey, or reconstituted whey powder), whey protein isolate (WPI), whey protein concentrate (WPC), a composition derived from any milk or colostrum processing stream, a composition derived from the retentate or permeate obtained by ultrafiltration or microfiltration of any milk or colostrum processing stream, a composition derived from the breakthrough or adsorbed extract obtained by chromatographic (including but not limited to ion and gel permeation chromatography) separation of any milk or colostrum processing stream, extracts of any of these milk derivatives including extracts prepared by multistage fractionation, differential crystallisation, solvent fractionation, supercritical fractionation, near critical fractionation, distillation, centrifugal fractionation, or fractionation with a modifier (e.g. soaps or emulsifiers), hydrolysates of any of these derivatives, extracts of the hydrolysates, and any combination of any two or more of these derivatives, including combinations of hydrolysed and/or non-hydrolysed extracts. It should be understood that the source of these derivatives may be milk or colostrum or a combination thereof.

In one embodiment a composition or product useful herein comprises, consists essentially of or consists of one or more agents as defined herein.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1 is a graph showing the dose response for IL-1beta when increasing the GMP dosage (μg/mL) in the THP-1 ELISA assay described in Example 1. GMP was provided by Fonterra Co-operative Group Limited, New Zealand.

FIG. 2 is a graph showing the dose response for IL-8 when increasing the G600™ milk fat extract dosage (μg/mL) in the THP-1 ELISA assay described in Example 1. G600™ milk fat extract was provided by Fonterra Co-operative Group Limited, New Zealand.

FIG. 3 is a graph showing the dose response for GMP (mg/kg) for the in vivo peritonitis model described in Example 2. Total cellular influx *p<0.05, **p<0.01, Dunnett's post test.

FIG. 4 is a graph showing the dose response for the G600™ milk fat extract (percentage of total fat) for the in vivo peritonitis model described in Example 2. Total cellular influx *p<0.05 Dunnett's post test.

FIG. 5 is a graph showing the dose response for the level of IL-1beta expression using RT-PCR from THP-1 assays with an increasing dosage of GMP (μg/mL) as discussed in Example 3.

FIG. 6 is a graph showing the dose response for the level of IL-8 expression using RT-PCR from THP-1 assays with an increasing dosage of the G600™ milk fat extract (μg/mL) as discussed in Example 3.

FIG. 7 is two graphs showing the change in serum urate levels in patients fed different dairy or soy products as discussed in Example 4 in terms of (A) change in concentration, and (B) percent change from baseline. The legend for graph (A) also relates to graph (B).

FIG. 8 is a graph showing fractional excretion of urate for patients fed different dairy or soy products as discussed in Example 4 (Tukey post test, late-season skim milk powder vs. soy p=0.126, late-season skim milk powder vs. early-season skim milk powder p=0.052, late-season skim milk powder vs. MPC 85™ skim milk powder p=0.02).

### DETAILED DESCRIPTION

The present invention relates to the discovery that glycomacropeptide (or a functional variant or fragment thereof) or gangliosides and phospholipids or a combination of glycomacropeptide, ganglioside and phospholipid can be used to treat or prevent gout, including gout flares. The glycomacropeptide, gangliosides and phospholipids, or combination thereof may usefully be administered in a dairy product, including a dairy product comprising less than about 4% lipid by weight.

The efficacy of a composition or method of the invention preferably comprises treating or preventing gout by maintaining or reducing inflammation caused by uric acid crystals or maintaining or reducing serum urate levels or both, optionally in combination with maintaining or increasing urate excretion in the urine. Additionally, a composition or method of the present invention may treat or prevent hyperuricaemia by maintaining or reducing serum uric acid levels.

### 1. Definitions

The term “beta-serum” means an aqueous dairy ingredient separated from dairy streams containing greater than 60% fat that have been through phase inversion from an oil-in-water to a water-in-oil emulsion, as described below. Cream is the preferred starting material for the production of beta-serum. For example, beta-serum is produced during the production of butter-oil (also known as anhydrous milk fat or AMF) from cream as shown in FIG. 2 of WO 2006/041316, incorporated herein by reference. Preferably the beta serum is dried; preferably dried beta-serum is a powder.

The term “comprising” as used in this specification means “consisting at least in part of”. When interpreting statements in this specification which include that term, the features, prefaced by that term in each statement or claim, all need to be present but other features can also be present. Related terms such as “comprise” and “comprised” are to be interpreted in the same manner.

An “effective amount” is the amount required to confer therapeutic effect. The interrelationship of dosages for animals and humans (based on milligrams per meter squared of body surface) is described by Freireich, et al. (1966). Body surface area can be approximately determined from height and weight of the subject. See, e.g., Scientific Tables, Geigy Pharmaceuticals, Ardley, N.Y., 1970, 537. Effective doses also vary, as recognized by those skilled in the art, dependent on route of administration, carrier usage, and the like.

The term “extract” means a composition that has been isolated from a source material and that is compositionally different to the source material that the extract was isolated from. For example, a mammalian milk extract, such as a sheep, goat, pig, mouse, water buffalo, camel, yak, horse, donkey, llama, or bovine milk extract; preferably a bovine milk extract, differs compositionally from the naturally occurring whole milk. In alternative embodiments the concentration in the extract is higher than the concentration in whole milk, or in whole colostrum, or in whey from milk, or in whey from colostrum, or in cream from milk, or in cream from colostrum, or in AMF from milk, or AMF from colostrum. Preferred source material useful herein includes whole milk or colostrum, skim milk or colostrum, or whey, cream, anhydrous milk fat, buttermilk, butter serum, beta serum, or whey cream from mammalian milk or colostrum, preferably milk from commercial dairy mammals, preferably bovine milk. Preferred extracts are lipid extracts, as described herein.

The term “functional fragment” is intended to mean a fragment of a polypeptide of SEQ ID NO. 1 or 2 that has activity when assayed according to the examples below. For example, without limitation, a functional fragment of SEQ ID NO: 1 or 2 may include a polypeptide of SEQ ID NO:1 or 2 when 1 to 15 amino acids are truncated from either the N terminus or the C terminus or when 1 to 15 amino acids are truncated from both the N terminus and the C terminus. Alternatively, a functional fragment of SEQ ID NO: 1 or 2 may include a truncation of from 1 to 30 amino acids at either the N terminus or the C terminus.

The term “functional variant” is intended to mean a variant of a polypeptide of SEQ ID NO. 1 or 2 that has activity when assayed according to the examples below.

The term “late season milk” means milk collected in the last quarter of the milking season, preferably from cows on a pastoral diet. Milk obtained from mammals fed on a diet comprising dry matter that results in milk comprising a similar compositional profile to late season milk may be used as a substitute. Additionally, milk produced using milking practices that result in milk comprising a similar compositional profile to late season milk may also be used as a substitute. The relevant compositional profile of late season milk is provided in the examples below, expressed as uric acid, orotic acid, purine and total purine in each dose of test material ingested. Seasonal variation of orotic acid and uric acid in milk is reported by Inyk and Woollard, 2004.

The term “milk fat extract” means an isolated extract of non-human Mammalian milk fat where the phospholipid and ganglioside concentration of the extract is higher than the phospholipid and ganglioside concentration of naturally occurring non-human mammalian milk fat. Preferably the concentration of at least one phospholipid and at least one ganglioside in an extract useful herein is at least about 0.5, 1, 5, 10, 15, 20, 25, 30, 35, 40, 45, 50, 55, 60, 65, 70, 75, 80, 85, 90, 95, or 100% higher than the concentration in naturally occurring non-human mammalian milk fat, and useful ranges may be selected between these values. In alternative embodiments the concentration in the extract is higher than the concentration in whole milk, or in whole colostrum, or in cream from milk, or in cream from colostrum, or in anhydrous milk fat (AMF) from milk, or AMF from colostrum.

The term “oral administration” includes oral, buccal, enteral and intra-gastric administration.

The term “pharmaceutically acceptable carrier” is intended to refer to a carrier including but not limited to an excipient, diluent, auxiliary or combination thereof that can be administered to a subject as a component of a composition of the invention that does not reduce the activity of the composition and is not toxic when administered in doses sufficient to deliver an effective amount of the active ingredient. The formulations can be administered orally, nasally and topically.

A “subject” is intended to mean an animal, preferably a mammal, more preferably a mammalian companion animal or human. Preferred companion animals include cats, dogs and horses.

The term “sweet whey” means an aqueous solution comprising whey proteins including glycomacropeptide, or a dried form thereof (including powders), wherein the sweet whey is produced by contacting milk with a milk clotting enzyme that can cleave the bond between phenylalanine and methionine at position 105 of kappa-casein. Suitable enzymes may include mammalian enzymes such as cow, sheep, goat, pig, chicken, or human enzymes, bacterial enzymes and plant enzymes. Such enzymes include but are not limited to rennet, chymosin, pepsin, or an acid proteinase from, for example, Rhizomucor meihei, Rhizomucor pusillus, or Cryphonectria parasitica, or a milk-clotting enzyme from, for example, Endothia parasitica, Bacillus polymyxa, Bacillus licheniformis, or Bacillus sphericus. A milk-clotting enzyme from certain plant species may also be used, for example from Withania coagulans, Ficus carica, pumpkin, seeds of Moringa oleifera, leaves of Calotropis procera, seeds of Ricinis communis, dried papaya latex, pineapple, cucumber, Benincasa cerifere, Galium verum, Pinquicule vulgaris, Drosera rotindifolia, Ranunculus ligua, Opuntia phylloclades (stem), Cereus triangularis (stem), Aloe L. sp. (stem), Euphorbia caducifolia (stem), Calotropis procera (leaf), Calotropis procera (flower), papaya (leaf), Ficus bengalensis (leaf), Ficus elastica (leaf), Euphorbia hista (whole plant) (see Dahot et al, 1990). Sweet whey may be dried by any means known in the art, including spray drying, freeze drying or evaporation.

The term “treat” and its derivatives should be interpreted in their broadest possible context. The term should not be taken to imply that a subject is treated until total recovery. Accordingly, “treat” broadly includes amelioration and/or prevention of the onset of the symptoms or severity of a particular condition such as, for example, gout or hyperuricaemia. Therefore, “treat” includes reducing gout specific inflammation, decreasing serum uric acid levels (including treating or preventing hyperuricaemia), and increasing urine uric acid excretion. In particular, and without wishing to be bound by theory, in one embodiment reducing gout specific inflammation may be achieved using an agent described herein such as GMP that modulates activation of the NALP-3 inflammasome resulting in the inhibition of processed IL-1beta.

The term “variant” refers to a naturally occurring (an allelic variant, for example) or non-naturally occurring (an artificially generated mutant, for example) polypeptide that varies from the amino acid sequence of SEQ ID NO. 1 or 2 by the addition, deletion or substitution of one or more amino acids. Naturally occurring variants of kappa-casein and GMP are described below.

Generally, a variant polypeptide possesses qualitative biological activity in common when assayed according to the examples below. Further, such variants may share at least about 80%, 85%, 90%, 95%, 96%, 97%, 98% or 99% sequence identity with the amino acid sequence of SEQ ID NO. 1 or 2. Also included within the meaning of the term “variant” are homologues of the amino acid sequence of SEQ ID NO. 1 or 2. A homologue is typically a polypeptide from a different species but sharing substantially the same biological function or activity as the corresponding polypeptide disclosed herein.

Preferred variant polypeptides preferably have at least about 80, 85, 90, 95 or 99% identity, preferably at least about 90, 95 or 99% identity to the amino acid sequence of SEQ ID NO. 1 or 2. Variant fragments preferably have at least about 80, 85, 90, 95 or 99% identity, preferably at least about 90, 95 or 99% identity to a fragment described herein. Identity can be determined by comparing a candidate amino acid sequence to a sequence described herein, such as an amino acid sequence of SEQ ID NO. 1 or 2 using the BLAST suite of programs (version 2.2.12; 28 Aug. 2005) that is publicly available from NCBI (ftp://ftp.ncbi.nih.gov/blast/).

Conservative substitutions of one or several amino acids of the amino acid sequence of SEQ ID NO. 1 or 2 without significantly altering its biological activity are also useful. A skilled artisan will be aware of methods for making phenotypically silent amino acid substitutions (see for example Bowie et al., (1990)).

### 2. Glycomacropeptide (GMP), Variants and Derivatives

Glycomacropeptide (GMP), also known as caseinomacropeptide (CMP) or caseinoglycomacropeptide (CGMP) is a fragment of the milk protein kappa-casein. Eleven genetic variants (A, B, C, D, E, F¹, F², G¹, G², H, I, and J) of bovine kappa-casein have been reported (Farrell et al, 2004, Table 2, incorporated herein by reference). Variants listed in Swiss-prot in the entry for kappa-casein (reference number P02668) include R-H at position 31 in variant F of P02668, R-C at position 118 in variant G, T-I at position 156 in variant G and variant H, T-I at position 157 in variant B and variant B2, D-A at position 169 in variant B and variant B2, I-T at position 174 in variant B2, and S-G at position 176 in variant E (position numbers refer to the kappa-casein peptide comprising the signal peptide, as discussed below). Kappa-casein is also present in the milk of other mammalian species, including human, sheep, goat, pig, mouse, water buffalo, camel, yak, horse, donkey, and llama milk, as described below, and use of GMP derived from the kappa-casein polypeptides of these species is also contemplated by the present invention.

Bovine kappa-casein (SEQ ID NO. 3, residues 1-169) may be hydrolysed into para-kappa-casein that comprises residues 1-105 of SEQ ID NO. 3 (SEQ ID NO. 4) and GMP that comprises residues 106-169 of SEQ ID NO. 3 (SEQ ID NOs: 1 and 2). Bovine GMP is known to exist in at least two forms, variant A that contains Thr at position 136 and Asp at position 148 of SEQ ID NO. 3 (SEQ ID NO. 1) and variant B that contains Ile at position 136 and Ala at position 148 of SEQ ID NO. 3 (SEQ ID NO. 2) (Brody, 2000; Thomä-Worringer, 2006). Other variants include those comprising Val at position 148, Gly at position 155 or Arg at position 155 (Farrell et al, 2004). These forms of GMP are contemplated by the current invention, as well as other reported sequence variants (Farrell et al, 2004). Accordingly, in one embodiment, the variant GMP comprises one or more of the amino acid substitutions T136I, D148A, D148V, S155G, and S155R of SEQ ID NO. 3.

In cheese making, para-kappa-casein is retained as the curd while GMP is removed with the whey, also known as “sweet whey”. GMP makes up 15-20% of the protein component of sweet whey and is around 8000 Da in size (Brody 2000). Hydrolysis of kappa-casein to produce GMP may be performed by any enzyme that can cleave the bond between phenylalanine and methionine at position 105 of bovine kappa casein; for example, proteinases or proteases, particularly acid proteinases or proteases that cleave the Phe Met bond at position 105 of bovine kappa casein. Enzymes that cleave at equivalent positions on the polypeptides of other species are known and may be substituted when non-bovine kappa-casein is used.

Hydrolysis of kappa-casein may be achieved using a milk clotting enzyme such as rennet, chymosin, pepsin, or an acid proteinase from, for example, Rhizomucor meihei, Rhizomucor pusillus, or Cryphonectria parasitica. Other suitable enzymes for producing GMP are discussed above in relation to production of sweet whey. The amino acid sequence of kappa-casein is reported with the accession number P02668 and NP_{—}776719. The 190 residue amino acid sequence of P02668/NP_{—}776719 comprises residues 22-190 of kappa-casein (SEQ ID NO. 3) and a 21 residue signal peptide (residues 1 to 21 of P02668/NP_{—}776719). The discussion in this specification of kappa-casein refers to the kappa-casein lacking the signal peptide. The nucleic acid sequence of bovine kappa-casein mRNA is reported with the accession number NM_{—}174294. Amino acid and nucleic acid sequences for kappa-casein polypeptides from other species have also been reported for human (CAA47048, X66417), sheep (AAS17943, AY444505), goat (AAM12027, AY090467), pig (NP_{—}001004026, NM_{—}001004026), mouse (NP_{—}031812, NM_{—}007786), water buffalo (AAU95771, AY750857), camel (CAA71171, Y10082), yak (AAM25909, AAM25910, AAF63191, AAB86616, AAB8661, AAB866177), horse (NP_{—}001075353, NM_{—}001081884) and donkey (ACA42445, EU448385) kappa-casein polypeptides.

GMP may be phosphorylated and glycosylated (Brody, 2000; Thomä-Worringer, 2006). Both the phosphorylated and non-phosphorylated forms of GMP are contemplated to be useful in the current invention. Additionally, both the glycosylated and non-glycosylated forms of GMP are contemplated to be useful in the present invention.

Glycomacropeptide is preferably isolated from mammalian milk, preferably bovine milk. GMP may also be produced recombinantly or synthetically from, or based on, the kappa-casein polypeptide or nucleic acid sequences described above, or from the polypeptide or nucleic acid sequences of other species. For example, GMP may be isolated from sheep, goat, camel, yak, llama, pig, horse, buffalo, or human milk, or from the milk of any other mammalian species whose milk contains kappa-casein. Equally, GMP may be produced recombinantly or synthetically from the bovine kappa-casein polypeptide or nucleic acid sequence of any of these species, or any other mammalian species that expresses kappa-casein. There are a number of ways of isolating GMP, for example the methodologies discussed and reported in Brody (2000) and Thomä-Worringer (2006), both incorporated herein by reference.

GMP may also be produced recombinantly using known techniques for the recombinant production of polypeptides (see for example Sambrook et al 1989, incorporated herein by reference in its entirety). A Bos taurus kappa-casein mRNA sequence has been reported under accession number NM_{—}174294. Preferred GMP polypeptide sequences that may be produced recombinantly include
(a) an isolated peptide consisting essentially of or consisting of SEQ ID NO. 1 or 2 or a functional fragment thereof,
(b) a functional variant of (a) having at least 80% sequence identity to SEQ ID NO. 1 or 2 or a functional fragment thereof,
(c) a peptide of about 64 to about 100 amino acids in length, wherein the peptide comprises SEQ ID NO. 1 or 2, and
(d) a functional variant of (c), wherein the peptide comprises an amino acid sequence having at least 80% sequence identity to SEQ ID NO. 1 or 2.

The current invention also contemplates the use of recombinant forms of glycomacropeptide from any of the above mentioned species, including recombinant human glycomacropeptide.

### 3. Milk Fat and Milk Fat Extracts

Milk fat is discussed comprehensively by Fox and McSweeney (2006), hereby incorporated by reference. In addition to lipids, milk fat includes vitamins, sterols, and minor components. See Chapter 1, Composition and Structure of Bovine Milk Lipids, Fox and McSweeney, for a description of naturally occurring bovine milk fat. Fractionation of milk fat is discussed in the Dairy Processing Handbook, 1995, and by Illingworth, 2002, and by Rombaut et al, 2006(b), all hereby incorporated by reference. Seasonal variation of milk fat is discussed by Fox and McSweeney (2006).

Examples of milk fat extracts useful according to the invention include cream (typically about 20 to about 40% fat by weight, preferably about 40% fat by weight), butter, ghee, anhydrous milk fat (AMF) (typically produced by phase inversion of cream or dehydration of butter), buttermilk, butter serum, beta serum, hard milk fat extracts, soft milk fat extracts, sphingolipid extracts, milk fat globule membrane extracts, milk fat globule membrane lipid extracts, phospholipid extracts, and complex lipid (lipids that yield 3 or more types of hydrolysis product per molecule) extracts, and combinations thereof, and hydrolysates thereof.

Buttermilk, butter serum, and beta serum are discussed by Bylund, 1995, Rombaut et al, 2005, Rombaut et al, 2006(a), Rombaut et al, 2006(b), and published international application WO 2006/041316, for example, all incorporated herein by reference. Buttermilk is a term used to describe the aqueous liquid phase obtained from traditional butter production using a butter making process which may be a batch (churn) process or a continuous (Fritz) process. Buttermilk is also a term used to describe the aqueous by-product produced by the cream concentration step of the traditional method of producing AMF from cream. This traditional method involves concentration then phase inversion of cream to produce oil that is further concentrated and polished to produce AMF. Finally, buttermilk is also a term used to describe a combination of the secondary skim and beta serum by-products of a two-serum process for AMF production—see for example, Bylund (Ed., 1995) and published international application WO 2006/041316 (see FIG. 2) that describe this process in detail. In that two-serum process, the by-product from the cream concentration step is further separated to produce secondary skim and the by-product from the oil concentration step is further separated to produce beta-serum. In the first two instances, the buttermilk is produced before any phase inversion has occurred. In the third instance, the buttermilk is a combination of secondary skim produced before phase inversion and beta serum produced after phase inversion. Concentration and polishing in these processes is typically achieved by centrifugation. Phase inversion is typically achieved by homogenisation. It should be understood that the source of these dairy lipid extracts may be milk or colostrum or a combination thereof.

Useful starting materials for fractionation include cream, AMF, butter milk, butter serum, or beta serum, from milk or colostrum or a combination thereof.

Multistage fractionation of milk fat may be carried out by differential crystallisation. Milk fat extracts are heated to a set temperature and the crystallised or solid (“stearin”—hard fraction) and liquid (“olein”—soft fraction) fractions are separated. Multi-step fractionation refers to re-fractionation in a subsequent step of a product of a previous fractionation step. Successive soft fractions may be produced by fractionating parent soft fractions into soft and hard sub-fractions.

Other fractionation methods include phase inversion, interesterification, glycerolysis, solvent fractionation (such as with ethanol, water, or acetone, used alone or sequentially), supercritical fractionation (see Astaire, et al, 2003, for example), near critical fractionation (see WO 2004/066744, for example), distillation, centrifugal fractionation, suspension crystallisation, dry crystallisation, fractionation with a modifier (e.g. soaps or emulsifiers), ultra-filtration, microfiltration, and any process for fractionation of lipid known in the art, and combinations of these methods, all as known in the art.

In one embodiment, the fractionation method is selected from solvent fractionation of cream, AMF, butter milk, butter serum, or beta serum, using ethanol, water, or acetone, alone or sequentially.

Lipids present in the compositions of the invention may be fully or partially modified, whether naturally, chemically, enzymatically, or by any other methods known in the art, including, for example, glycosylated, sialylated, esterified, phosphorylated or hydrolysed. Lipid hydrolysates may be prepared using known techniques, including but not limited to acid hydrolysis, base hydrolysis, enzymatic hydrolysis using a lipase, for example as described in Fox and McSweeney ((2006), Chapter 15 by H C Deeth and C H Fitz-Gerald), and microbial fermentation. One method of base hydrolysis includes adding 1% KOH (in ethanol) and heating for 10 minutes. Hydrolysed material may be neutralised with acetic acid or hydrochloric acid.

Milk fat globule membrane material may be isolated according to the acidification method of Kanno & Dong-Hyun, 1990, and further fractionated into lipid and protein fractions by the addition of methanol, as described by Kanno et al, 1975. A phospholipid extract may be isolated by extracting the lipid mixture with acetone according to the procedure of Purthi et al, 1970. Lipid residue may be further enriched in milk fat globule membrane lipids by the selective extraction of non-polar lipids with pentane.

Fractionation methods useful to produce milk fat extracts useful herein are also described in published international patent applications WO 2006/041316, WO 2007/123424, and WO 2007/123425 that are each hereby incorporated herein by reference in their entirety.

Particularly preferred milk fat extracts useful herein includes those described in the examples below and those summarised in the following Tables 1a and 1b. These extracts may be dried, and may be powders, optionally with components including flow aids such as lactose added to improve flowability. Fraction 1 is beta-serum. Fractions 2, 3, 4 and 5 are prepared by ethanol extraction of beta-serum powder. Beta serum is the liquid phase produced during AMF manufacture. The fractions including beta-serum, the G600™ milk fat precursor (Batch 1 and Batch 2, a manufacturing precursor to the G600™ milk fat extract), the G500™ milk fat extract, and the G600™ milk fat extract were obtained from Fonterra Co-operative Group Limited, New Zealand. Fractions 6 to 9 described in Table 1b below may be produced according to the methods described in published international patent application WO 2006/041316 (see examples 3 to 6). Fraction 10 may be produced by supercritical carbon dioxide extraction of Fraction 8.

TABLE 1a
Milk fat extracts useful herein

| | Fraction | | | | |
|---|---|---|---|---|---|
| | | 2 | 4 | | |
| | 1 | G600 ™ | G600 ™ | 3 | 5 |
| Component | Beta | precursor | precursor | G500 ™ | G600 ™ |
| (% w/w) | serum | (Batch 1) | (Batch 2) | extract | extract |
| Protein | 30.2 | ND | 10 | <2% | 10.2 |
| MFGM | 7.5 | ND | ND | ND | ND |
| Fat | 20.6 | 84.6 | 73 | 35.5 | 27.9 |
| Phospholipid | 9.7 | 27.6 | 44 | 17.6 | 15.1 |
| PC | 2.5 | 3.2 | 5.8 | 3.1 | 2.0 |
| PI | 0.8 | 6.0 | 8.4 | 2.8 | 2.9 |
| PS | 1.1 | 7.3 | 11.6 | 3.5 | 4.0 |
| PE | 2.8 | 6.4 | 12.7 | 4.9 | 4.4 |
| SM | 2.4 | 3.5 | 4.6 | 2.8 | 1.6 |
| Gangliosides | 0.4 | 4.5 | 5.8 | 1.3 | 2.0 |
| GD3 | 0.4 | 4.0 | 5.2 | 0.6 | 1.8 |
| Lactose | ND | 8.3 | 14 | 54.9 | 58.0 |
| Ash | ND | 7.0 | 10 | 5.0 | 8.3 |
| Moisture | 1.9 | 3.7 | 3 | 3.2 | 2.8 |

ND = not determined;
% w/w = % by weight.

TABLE 1b
Milk fat extracts useful herein

| Component | Fraction | | | | |
|---|---|---|---|---|---|
| (% w/w) | 6 | 7 | 8 | 9 | 10 |
| Protein | 49.7 | 60.2 | <0.01 | <0.01 | 12.4 |
| MFGM | 11.9 | 14.4 | 0.2 | ND | ND |
| Fat | 35.6 | 23.1 | 94.2 | 86.8 | 90.2 |
| Phospholipid | 14.9 | 16.0 | 31.0 | 65.7 | 66.8 |
| PC | 3.8 | 4.9 | 8.1 | 16.8 | 15.0 |
| PI | 1.1 | 1.5 | 2.8 | 5.8 | 6.0 |
| PS | 1.6 | 2.1 | 4.3 | 8.7 | 7.6 |
| PE | 4.3 | 5.4 | 11.3 | 23.6 | 21.8 |
| SM | 3.6 | 4.5 | 7.5 | 16.5 | 13.6 |
| Gangliosides | 0.7 | 1.0 | 1.2 | 2.0 | 2.0 |
| GD3 | 0.6 | 0.9 | 1.1 | 1.8 | 1.8 |
| Lactose | 7.8 | 11.7 | 2.6 | 6.4 | 4.0 |
| Ash | 5.2 | 5.9 | 3.1 | 12.1 | 9.1 |
| Moisture | 2.7 | 2.9 | 2.6 | 4.6 | 2.3 |

ND = not determined;
<0.01 = trace amounts

The G500™ milk fat extract is a spray dried milk ganglioside concentrate to which lactose has been added to improve powder flowability. The G500™ milk fat extract has a typical fatty acid composition of myristic acid (14:0) 5.6%, palmitic acid (16:0) 18.4%, palmitoleic acid (16:1) 1.2%, margaric acid (17:0) 0.5%, stearic acid (18:0) 14.9%, oleic acid (18:1) 31.0%, linoleic acid (18:2) 3.8%, linolenic acid (18:3) 1.5%, and arachidonic acid (20:4) 0.5%. The G600™ milk fat extract is a spray dried milk ganglioside concentrate to which lactose has been added to improve powder flowability. The G600™ milk fat extract has a typical fatty acid composition of myristic acid (14:0) 4.7%, palmitic acid (16:0) 16.4%, palmitoleic acid (16:1) 1.2%, margaric acid (17:0) 0.5%, stearic acid (18:0) 17.0%, oleic acid (18:1) 33.4%, linoleic acid (18:2) 4.2%, linolenic acid (18:3) 1.4%, and arachidonic acid (20:4) 0.6%. Before addition of lactose, the G500™ milk fat extract and the G600™ milk fat extract are useable as precursors, with or without drying such as freeze-drying or spray-drying and without added lactose.

In the fractions described above, protein levels were determined by total nitrogen multiplied by 6.38. Phospholipid levels were determined by ³¹P NMR. Ganglioside levels were determined as follows. In triplicate, approximately 0.1 g of powder was weighed into a 16 nil kimax tube and the weight recorded. 6 ml of methanol was added and mixed by vortexing for 1 min. The solution was incubated at 50° C. for 10 min then 6 ml water was added and mixed by vortexing. The solution was allowed to stand for 2 hrs at 4° C. to settle and a sample was taken and passed through a 0.45 μm filter. The sample was analysed by HPLC. A Cosmosil™ 5NH2-MS waters column (Nacalai Tesque Inc, USA) was used with a NH2 security guard (Phenomenex™ AJO-4302 in a Phenomenex™ KJO-4282 holder). The guard cartridge was changed every day of analysis. Injections of sample were injected onto the column and eluted at a flow rate of 2 ml/min using solvent A (90% acetonitrile, 5% water and 5% 5 mM phosphate buffer pH 5.6) and solvent B (50% acetonitrile, 45% water and 5% 200 mM phosphate buffer pH5.6). The following Gradient was used: 100% A for 3.5 min, then 100% A to 55% A over 26.5 min, then 55% A to 100% A over 1 min and then 100% A for 5 min (Wagener et al. (1996), Journal of Lipid Research 37, 1823-1829). An external standard curve of 0-2 ug GD3 was generated using buttermilk GD3 (Matreya #1504). Elution was monitored at 203 nm.

### 4. Compositions Useful According to Invention

A composition useful herein may be formulated as a food, drink, food additive, drink additive, dietary supplement, nutritional product, medical food, enteral or parenteral feeding product, meal replacement, cosmeceutical, or pharmaceutical. Appropriate formulations may be prepared by an art skilled worker with regard to that skill and the teaching of this specification.

In one embodiment, compositions useful herein include any edible consumer product which is able to carry lipid or protein or both. Examples of suitable edible consumer products include powders, liquids, confectionary products including chocolate, gels, ice creams, reconstituted fruit products, snack bars, food bars, muesli bars, spreads, sauces, dips, dairy products including yoghurts and cheeses, drinks including dairy and non-dairy based drinks (such as milk drinks and yogurt drinks), milk powders, sports supplements including dairy and non-dairy based sports supplements, food additives such as protein sprinkles and dietary supplement products including daily supplement tablets. Suitable nutraceutical compositions useful herein may be provided in similar forms.

In alternative embodiments, the compositions useful herein may be formulated to allow for administration to a subject by any chosen route, including but not limited to oral or parenteral (including topical, subcutaneous, intramuscular and intravenous) administration.

Thus, a pharmaceutical composition useful according to the invention may be formulated with an appropriate pharmaceutically acceptable carrier (including excipients, diluents, auxiliaries, and combinations thereof) selected with regard to the intended route of administration and standard pharmaceutical practice. For example, a composition useful according to the invention can be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard shell tablet or a capsule containing a binder, e.g., lactose or mannitol, a conventional filler, and a tabletting agent. Pharmaceutical compositions can also be administered via the parenteral route. Examples of parenteral dosage forms include aqueous solutions, isotonic saline or 5% glucose of the active agent, or other well-known pharmaceutically acceptable excipients. Cyclodextrins, or other solubilising agents well-known to those familiar with the art, can be utilized as pharmaceutical excipients for delivery of the therapeutic agent.

The efficacy of a composition useful according to the invention can be evaluated both in vitro and in vivo. See, e.g., the examples below. Briefly, the composition can be tested in vitro or in vivo for its ability to reduce an inflammatory response to uric acid crystals. For in vivo studies, the composition can be fed to or injected into an animal (e.g., a mouse) and its effects on inflammation, pain, serum uric acid levels and urate excretion are then assessed. Based on the results, an appropriate dosage range and administration route can be determined.

The compositions useful herein may be used alone or in combination with one or more other therapeutic agents. The therapeutic agent may be a food, drink, food additive, drink additive, food component, drink component, dietary supplement, nutritional product, medical food, nutraceutical, medicament or pharmaceutical. The therapeutic agent is preferably effective to attenuate one or more of the symptoms of gout or gout flares, including one or more of pain, inflammation and serum uric acid levels.

When used in combination with another therapeutic agent, the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agent are provided.

Suitable agents with which the compositions useful herein can be co-administered include known agents for treating or preventing gout including but not limited to uricosuric agents that increase the excretion of uric acid in the urine thereby reducing serum uric acid concentrations (for example, probenecid, benzbromarone, sulfinpyrazone, guaifenesin, and losartan) and anti-inflammatory agents.

It should be understood that the additional therapeutic agents listed above (both food based and pharmaceutical agents) may also be employed in a method according to the invention where they are administered separately, simultaneously or sequentially with a composition useful herein.

As will be appreciated, the dose of the composition administered, the period of administration, and the general administration regime may differ between subjects depending on such variables as the severity of symptoms of a subject, the type of disorder to be treated, the mode of administration chosen, and the age, sex and/or general health of a subject. However, by way of general example, the inventors contemplate administration of from about 1 mg to about 1000 mg per kg body weight of a milk fat extract useful herein per day, preferably about 1 to about 500 mg/kg/day, alternatively about 150 to about 410 mg/kg/day, alternatively about 1 to about 100 mg/kg/day or about 1 to about 20 mg/kg/day. The inventors also contemplate administration of from about 1 mg to about 1000 mg per kg body weight of a GMP per day, preferably about 1 to about 500 mg per kg per day, alternatively about 1 to about 100 mg/kg/day or about 1 to about 30 mg/kg/day. In one embodiment, the inventors contemplate administration of from about 0.05 mg to about 250 mg per kg body weight of a pharmaceutical composition useful herein.

It should be appreciated that administration may include a single daily dose or administration of a number of discrete divided doses as may be appropriate.

Various aspects of the invention will now be illustrated in non-limiting ways by reference to the following examples.

### EXAMPLES

### Example 1

### THP-1 Cell Assay Analysed by ELISA

THP-1 cells were cultured in RPMI with 10% FCS, penicillin, streptomycin, glutamine and pyruvate. At day −1, 1.5×10⁶ THP-1 cells were plated in 24 well plates. These cells were cultured in the presence or absence of 5 μM phorbol 12-myristate 13-acetate (PMA) for three hours. PMA is required for NALP-3 inflammasome activation and IL-1β production in response to MSU crystals. On day 0, medium was replaced in all wells. Cells were then pre-incubated with vehicle control, test substance at three concentrations, or diclofenac control (10 μg/ml) for 4 hours. MSU crystals were then added (0.5 mg/mL) Supernatants were harvested for ELISA after 16 hours. ELISA was conducted to determine inhibition of IL-1β expression. It has been reported that this cytokine is a pivotal regulator of the inflammatory response to MSU crystals (Chen et al 2006 and Martinon et al 2006). Inhibition of IL-8 expression was also determined.

A mean change in cytokine expression from cells treated with control vehicle pooled data from all experiments (10 μg/ml). Values represent mean (standard error) from at least three experiments in which cytokine release from wells treated with the highest concentration of milk extract has been normalized to vehicle treated wells.

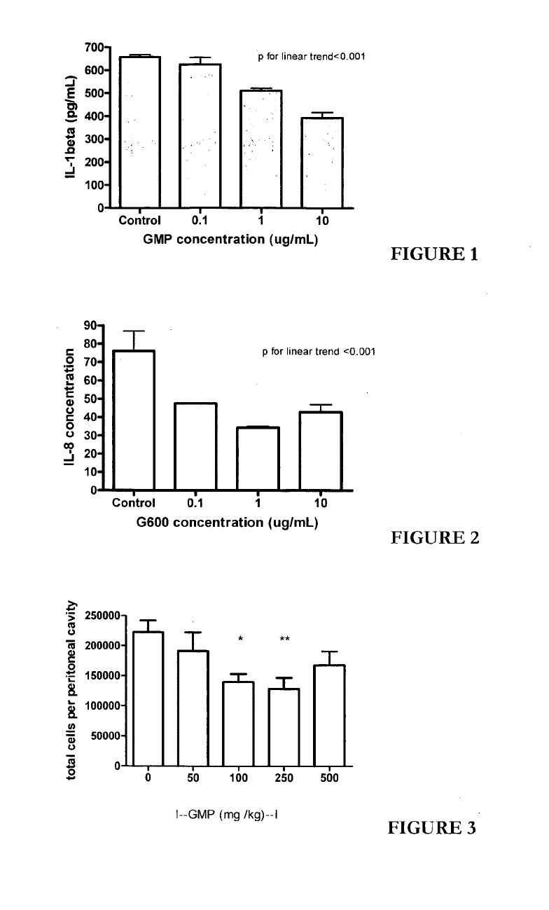

Test substances (provided by Fonterra Co-operative Group Limited, New Zealand) which showed significant reduction in the release of the measured cytokines are indicated by (*), p<0.05 compared with the corresponding vehicle control. The results are shown in Table 2 below and in FIGS. 1 and 2. Table 3 contrasts the result for GMP when the MSU was replaced with a different antigen, LPS. The casein hydrolysate tested was produced by extensively hydrolysing casein (to a degree of hydrolysis of 33%) using the enzymes pancreatin and corolase. The casein hydrolysate did not contain GMP. The PC500™ milk fat extract is a phospholipid extract prepared by ethanol extraction of beta-serum that comprises about 87% total lipid and 37% phospholipid by weight but no detectable gangliosides.

TABLE 2
Results of Example 1

| Test substance | IL-1β | IL-8 |
|---|---|---|
| GMP | 0.72* | 1.22 |
| Casein hydrolysate | 0.84 | 1.23 |
| PC500 ™ milk fat extract | 0.84 | 0.82 |
| G600 ™ milk fat extract | 0.81 | 0.72* |

TABLE 3
Result for GMP when MSU replaced with LPS

| Test | MSU crystal-stimulated IL-1β | LPS-stimulated IL-1β |
|---|---|---|
| substance | production | production |
| GMP | 0.72* | 0.94 |

### Example 2

### Peritonitis Assays

Adult male CD1 mice (Vernon Jansen Unit, University of Auckland) were housed in conventional facilities with temperature control and 12 hour light/dark cycles. All protocols involving live animals were subjected to ethical review and a range of welfare score sheets and action plans were used to monitor and minimize the impact of the study on the animals. Mice were fed milk extracts for a week prior to induction of peritonitis. At least eight mice were used for each experimental condition. GMP was dissolved in drinking water at 0.05, 0.1, 0.25 and 0.5 g per kg mouse. The G600™ milk fat extract precursor (Batch 2, freeze dried, no added lactose) was incorporated into rodent chow at 12.5, 25, 50 and 100% of total fat in diet.

The murine urate peritonitis protocol was based on the model described by Getting et al (1997). Adult male CD1 mice weighing 22-28 g were selected for the urate peritonitis model. Mice were anaesthetized using urethane. Peritonitis was induced by intraperitoneal injection of 3 mg MSU crystals resuspended in 250 μl PBS. The body temperature of anaesthetized mice was monitored and maintained at 37° C. using a heat pad, and hydration was maintained by subcutaneous injection of 0.9% NaCl (Normal Saline) at a volume of 10 ml/g body weight two hours after initial treatments and every hour thereafter. After four hours exposure to MSU crystals the mice were sacrificed by rising concentrations of CO₂, and 3 ml PBS containing 25 U/ml heparin was injected into each peritoneal cavity. The peritoneal lavage fluid was then aspirated, and 500 μl aliquots of lavage fluid were centrifuged for flow cytometry (FACS) analysis for the total number of cells and the number of neutrophils.

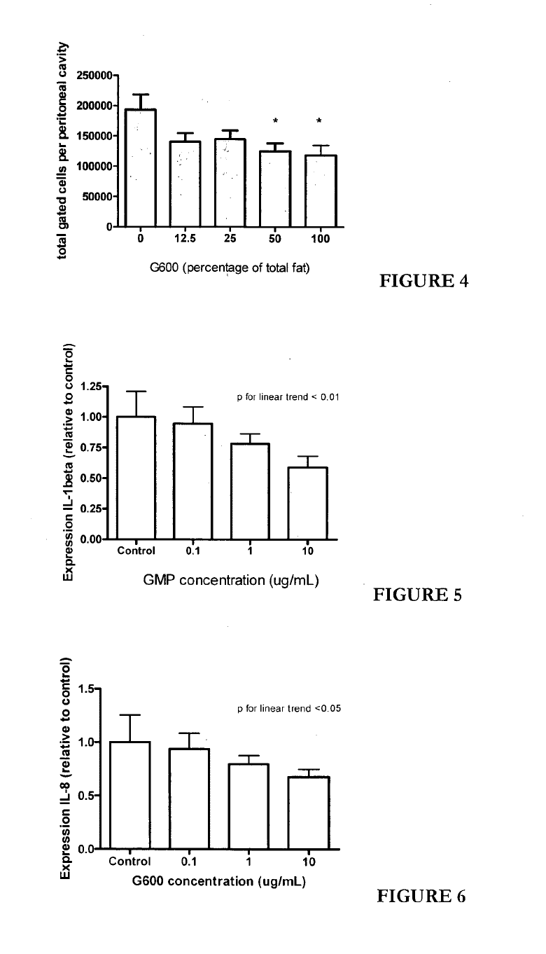

The GMP results for the peritonitis in vivo assays are shown in FIG. 3. The results for the G600™ milk extract precursor (Batch 2) are shown in FIG. 4 (one way ANOVA with Dunnett's post test *p<0.05, **p<0.01).

In summary, both GMP and the G600™ milk extract precursor (Batch 2, freeze-dried, no added lactose) led to a significant inhibition in total cellular influx in the urate peritonitis model.

### Example 3

### THP-1 Cell Assay Analysed by RT-PCR

THP-1 assays were used to study IL-1β and IL-8 gene expression by RT-PCR, using the same treatment protocols as described for the THP-1 ELISA assay in Example 1 above and the RT-PCR protocol described in Dalbeth et al, 2008.

These assays showed that GMP inhibits IL-1β gene expression (FIG. 5) and the G600™ milk extract precursor (Batch 2) inhibits IL-8 gene expression (FIG. 6) in THP-1 cells.

### Example 4

### Human Study

The control material was soy milk powder (ProFam 873™ from Archer Daniels Midland, USA) and the test materials were early-season skim milk powder (August), late-season skim milk powder (March, April, May), and MPC85™ milk protein concentrate skim milk powder. All skim milk and milk protein concentrate powders were obtained from Fonterra Co-operative Group Limited, New Zealand. Table 4 shows the amount of uric acid, orotic acid, purine and total purine in each dose of test material ingested. Seasonal variation of orotic acid and uric acid in milk is reported by Inyk and Woollard, 2004.

TABLE 4
Test material composition

| | Uric acid | Orotic acid | Purine | Total purine |
|---|---|---|---|---|
| Test Material | (mg) | (mg) | (mg) | (mg)^{#} |
| MPC | 2.45 | 3.92 | 0.075 | 2.525 |
| SMP Late season | 42.77 | 171.7 | 0.47 | 43.24 |
| SMP early season | 60.95 | 75.44 | 0.69 | 61.64 |
| Soy protein | 0 | 0 | 36.26 | 36.26 |
| isolate | | | | |

^{#}total purine is uric acid plus purine value

16 healthy male participants took part in this study, with the primary endpoint of change in serum urate concentration at three hours. Participants ingested an amount of the control or test substances equal to 80 g protein on separate visits. These substances were selected for their varying purine and orotic acid composition.

Each substance was administered as 80 g protein in an 800 mL suspension. Participants received one of the test substances over a five week period, but substances were administered in a random order. Participants and investigators were blinded to the substances. The substances were suspended using a bar-mixer (wand) and 800 ml tap water (potable) at about 50° C. The liquid was then refrigerated for ingestion within 24 hours.

### Study Visit Summary

At each study visit, a venous catheter was inserted for blood collection. Following an overnight fast, subjects consumed the substances of interest between 0800 and 0900, and blood was obtained to test urate, creatinine, urea, albumin, total protein and serum storage, prior to ingestion and then 60 minutes, 120 minutes, and 180 minutes after ingestion. Urine volume was measured and urine was obtained at these time points for testing of uric acid and creatinine. This was a cross-over design with the same subjects returning every one to two weeks for testing (up to eight weeks).

### Change in Serum Urate Concentrations

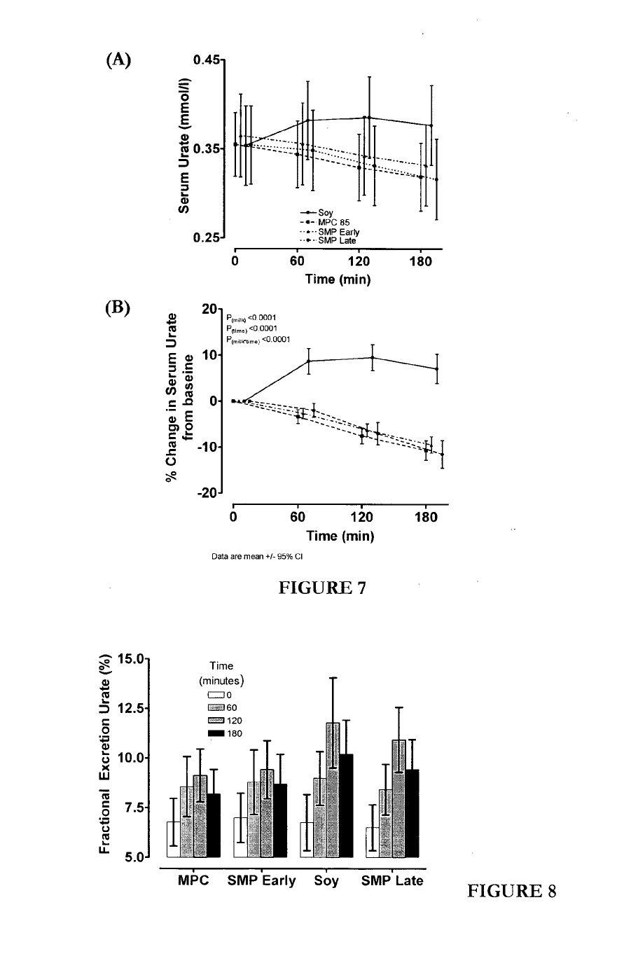

All three skim milk powders reduced serum urate concentrations by approximately 10% over three hours. This was in contrast to the soy milk powder which led to a 10% increase in serum urate concentration. All three skim milk powders inhibited serum urate concentrations by a similar magnitude. These results are shown in FIGS. 7 (A) and (B).

### Fractional Excretion of Urate

All substances tested had a uricosuric effect, with highest rates of uric acid excretion evident in the soy product and late-season skim milk powder (Tukey post test, late-season skim milk powder vs. soy p=0.126, late-season skim milk powder vs. early-season skim milk powder p=0.052, late-season skim milk powder vs. MPC 85™ skim milk powder p=0.02). These results are shown in FIG. 8.

### Example 5

### Gout Flares

Patients with recurrent gout flares are randomized to three separate groups and a control or treatment product is administered daily as a shake. The three products are a control lactose powder shake, a standard skim milk powder shake, and a skim milk powder shake enriched with GMP and G600™. The primary endpoint is frequency of gout flares, and secondary endpoints are serum urate concentration, fractional excretion of urate, and C-reactive protein.

### 1. Patient Selection

Patients are identified from a rheumatology clinic and inpatient databases throughout Auckland, and through advertising to general practitioners and the public.

The patient inclusion criteria requires that the patient:
has been diagnosed with gout (based on American College of Rheumatology criteria for diagnosis of gout),
has had at least two gout flares in the last four months,
is able to provide written informed consent, and
is ≧18 years of age.

Patients with lactose intolerance and/or severe renal impairment are excluded.

### 2. Test Compositions

Patients are randomized to one of three products for three months:
Control—lactose powder,
Skim milk powder (late production) 15 g protein, or
Standard skim milk powder (late production) enriched with GMP and G600™ (1.5 g GMP (10% total protein), and 0.525 g G600™ (3.5%)), 15 g protein.

Each product will be administered daily as a 250 mL vanilla flavoured shake.

Compliance will be assessed by sachet count returns.

### 3. Assessment Procedures

### 3.1. Primary Endpoint

Frequency of gout flares requiring treatment using a gout flare diary is measured at 0, 1, 2, and 3 months. Gout flares requiring treatment are determined by self-report with use of pain visual analogue scale score>3 to verify.

### 3.2. Secondary Endpoint

Severity of flare pain (10-point Likert scale), using a gout flare diary
Length of flare, using a gout flare diary
Serum urate concentration
Fractional excretion of urate
Swollen and tender joint counts
C-reactive protein
Erythrocyte sedimentation rate (ESR)
Patient global assessment using a 5-point Likert scale
Functional assessment (Health Assessment Questionnaire)
Number of patients requiring treatment for flare

### INDUSTRIAL APPLICATION

The present invention has utility in treating or preventing gout. The described compositions may be employed as foods, drinks, food additives, drink additives, dietary supplements, nutritional products, medical foods, nutraceuticals, medicaments or pharmaceuticals.

Those persons skilled in the art will understand that the above description is provided by way of illustration only and that the invention is not limited thereto.

### REFERENCES

Astaire J. C., Ward R., German J. B., and Jimenez-Flores R. (2003) Concentration of Polar MFGM Lipids from Buttermilk by Microfiltration and Supercritical Fluid Extraction J. Dairy Sci. 86, 2297-2307
Bowie J U, Reidhaar-Olson J F, Lim W A, Sauer R T. Deciphering the message in protein sequences: tolerance to amino acid substitutions. Science. (1990) 247(4948):1306-10. Brody E., (2000) Biological activities of bovine glycomacropeptide British Journal of Nutrition 84, S39-S46.
Bylund, G. (Ed.) Dairy processing handbook. (1995) Tetra Pak Processing Systems AB, S-221 86 Lund, Sweden.
Campion E W, Glynn R J, DeLabry L O. Asymptomatic hyperuricemia. Risks and consequences in the Normative Aging Study. Am J Med. 1987 82(3):421-426.
Chen C J, Shi Y, Hearn A, Fitzgerald K, Golenbock D, Reed G, et al. MyD88-dependent IL-1 receptor signaling is essential for gouty inflammation stimulated by monosodium urate crystals. J Clin Invest 2006; 116(8):2262-71.
Dahot M U, Khan M Y, Memon A N, Screening Of Some Pakistani Plants For Milk Clotting Activity, Journal of Islamic Academy of Sciences 3:4, 284-286, 1990
Dalbeth N., Smith T., Gray S., Doyle A., Antill P., Lobo M., Robinson E., King A., Cornish J., Shalley G., Gao A., McQueen F M., Cellular characterization of magnetic resonance imaging (MRI) bone edema in rheumatoid arthritis; implications for pathogenesis of erosive disease. Ann Rheum Dis. 2008 Sep. 2; 18765428
Farrell H M Jr et al, Nomenclature of the proteins of cows' milk—sixth revision, J Dairy Sci. 2004 June; 87(6):1641-74
Fox P F, McSweeney P L H (eds), Advanced Dairy Chemistry, Volume 2—Lipids, 3rd Ed, Springer Science+Business Media, Inc., 2006.
Freireich E J, Gehan E A, Rall D P, Schmidt L H, Skipper H E (1966) Quantitative comparison of toxicity to anticancer agents in mouse, rat, hamster, dog, monkey and man. Cancer Chemother Rep 50: 219-244.
Getting S J, Flower R J, Parente L, De Medicis R, Lussier A, Woliztky B A, et al. Molecular determinants of monosodium urate crystal-induced murine peritonitis: A role for endogenous mast cells and a distinct requirement for endothelial-derived selectins. Journal of Pharmacology and Experimental Therapeutics 1997; 283(1):123-130.
Gout in primary care. Drug therapeutics bulletin 42(5): 37-40 (2004)
Indyk H E, Woollard D C, Determination of Orotic Acid, Uric Acid, and Creatinine in Milk by Liquid Chromatography, J. AOAC Int. 2004 87(1):116-122
Kanno C, Shimizu M & Yamachi K (1975). Isolation and physiochemical properties of a soluble glycoprotein fraction of milk fat globule membrane. Agric. Biol. Chem., 39(9):1835-1842.
Kanno C & Dong-Hyun K (1990). A simple procedure for the preparation of bovine milk fat globule membrane and a comparison of its composition, enzymatic activity, and electrophoretic properties with these prepared by other methods. Agric. Biol. Chem., 54(11):2845-2854.
Martinon F, Petrilli V, Mayor A, Tardivel A, Tschopp J. Gout-associated uric acid crystals activate the NALP3 inflammasome. Nature 2006; 440(7081):237-41.
Martinon and Glimcher (2006). Gout: new insights into an old disease. The Journal of Clinical Investigation, 116:2073-2075.
Pruthi T D, Narayanan K M & Bhaleerao V R (1970). The role of milk phospholipids in the autoxidation of butterfat—I. Indian Journal of Dairy Science, 23:248-251
Roddy E,. Zhang W., Doherty M., The changing epidemiology of gout. Nature Clinical Practice. (2007) 3(8): 443-449.
Rombaut R, Camp J V, Dewettinck K., Analysis of phospho- and sphingolipids in dairy products by a new HPLC method. J Dairy Sci. (2005) 88(2):482-8.
Rombaut R, Dejonckheere V, Dewettinck K., Microfiltration of butter serum upon casein micelle destabilization. J Dairy Sci. (2006)(a) 89(6):1915-25.
Rombaut R., Van Camp J. & Dewettinck K., Phospho- and sphingolipid distribution during processing of milk, butter and whey, International Journal of Food Science & Technology, (2006)(b) 41(4):435-443.
Sambrook, J.; Fritsch, E. F.; Maniatis, T. (1989). Molecular Cloning: A Laboratory Manual. Cold Spring Harbour Lab Press, Cold Spring Harbour, N.Y.
C. Thomä-Worringer et al., Health effects and technological features of caseinomacropeptide, International Dairy Journal 16 (2006) 1324-1333
Underwood M., (2006) Clinical review, Diagnosis and management of gout, BMJ, 332: 1315-1319.

1.-29. (canceled)

30. A method for treating or preventing gout comprising administering an effective amount of one or more agents to a subject in need thereof, wherein the one or more agents are selected from:
a. about 0.1 to about 99.9% by weight phospholipid and about 0.1 to about 99.9% by weight ganglioside,
b. a milk fat extract comprising about 0.1 to about 99.9% by weight phospholipid and about 0.1 to about 99.9% by weight ganglioside,
c. a milk fat extract comprising about 1 to about 90% lipid wherein the lipid comprises about 0.1 to about 99.9% by weight phospholipid and about 0.1 to about 99.9% by weight ganglioside, and
d. any combination of any two or more of (a) to (c).

31. A method of claim 30, further comprising administering an effective amount of one or more additional agents to a subject in need thereof, wherein the one or more additional agents is selected from:
a. glycomacropeptide,
b. an isolated peptide consisting essentially of or consisting of SEQ ID NO. 1 or 2 or a functional fragment thereof,
c. a functional variant of (b) having at least 80% sequence identity to SEQ ID NO. 1 or 2 or a functional fragment thereof,
d. a peptide of about 64 to about 100 amino acids in length, wherein the peptide comprises SEQ ID NO. 1 or 2,
e. a functional variant of (d), wherein the peptide comprises an amino acid sequence having at least 80% sequence identity to SEQ ID NO. 1 or 2,
f. a casein hydrolysate comprising one or more peptides consisting of SEQ ID NO. 1 or 2, wherein the hydrosylate comprises about 51 to about 100% by weight of the one or more peptides,
g. sweet whey, and
h. any combination of any two or more of (a) to (g).

32. A method of claim 31, wherein the one or more agents and the one or more additional agents are administered separately, simultaneously or sequentially.

33. A method of claim 31, comprising the administration of a composition comprising about 51 to about 99% by weight of the one or more additional agents as defined in claim 2.

34. A method of claim 30, comprising the administration of a composition that comprises about 1% to about 25% by weight phosphatidylcholine, about 0.1% to about 15% by weight phosphatidylinositol, about 0.1% to about 15% by weight phosphatidylserine, about 1% to about 30% by weight phosphatidylethanolamine, and about 0.5% to about 25% by weight sphingomyelin.

35. A method of claim 30, comprising the administration of a composition that comprises about 0.1% to about 30% by weight of one or more gangliosides.

36. A method of claim 35, wherein the composition comprises about 0.1% to about 5% by weight of one or more gangliosides.

37. A method of claim 30, wherein the agents are administered in a dairy product.

38. A method of claim 37, wherein the dairy product increases uric acid excretion.

39. A method of claim 31, comprising the administration of a composition that comprises about 1% to about 25% by weight phosphatidylcholine, about 0.1% to about 15% by weight phosphatidylinositol, about 0.1% to about 15% by weight phosphatidylserine, about 1% to about 30% by weight phosphatidylethanolamine, and about 0.5% to about 25% by weight sphingomyelin.

40. A method of claim 31, comprising the administration of a composition that comprises about 0.1% to about 30% by weight of one or more gangliosides.

41. A method of claim 40, wherein the composition comprises about 0.1% to about 5% by weight of one or more gangliosides.

42. A method of claim 31, wherein the agents are administered in a dairy product.

43. A method of claim 42, wherein the dairy product increases uric acid excretion.

44. A composition comprising one or more agents selected from:
a. about 0.1 to about 99.9% by weight phospholipid and about 0.1 to about 99.9% by weight ganglioside,
b. a milk fat extract comprising about 0.1 to about 99.9% by weight phospholipid and about 0.1 to about 99.9% by weight ganglioside,
c. a milk fat extract comprising about 1 to about 90% lipid wherein the lipid comprises about 0.1 to about 99.9% by weight phospholipid and about 0.1 to about 99.9% by weight ganglioside, and
d. any combination of any two or more of (a) to (c);
and wherein the composition further comprises one or more additional agents selected from:
e. glycomacropeptide,
f. an isolated peptide consisting essentially of or consisting of SEQ ID NO. 1 or 2 or a functional fragment thereof,
g. a functional variant of (b) having at least 80% sequence identity to SEQ ID NO. 1 or 2 or a functional fragment thereof,
h. a peptide of about 64 to about 100 amino acids in length, wherein the peptide comprises SEQ ID NO. 1 or 2,
i. a functional variant of (d), wherein the peptide comprises an amino acid sequence having at least 80% sequence identity to SEQ ID NO. 1 or 2,
j. a casein hydrolysate comprising one or more peptides consisting of SEQ ID NO. 1 or 2, wherein the hydrosylate comprises about 51 to about 100% by weight of the one or more peptides,
k. sweet whey, and
l. any combination of any two or more of (e) to (k).

45. A composition of claim 44 that is a dairy product.

46. A composition of claim 45, wherein the dairy product increases uric acid excretion.

47. A method for treating or preventing gout comprising administering an effective amount of one or more agents to a subject in need thereof, wherein the one or more agents is selected from:
a. glycomacropeptide,
b. an isolated peptide consisting essentially of or consisting of SEQ ID NO. 1 or 2 or a functional fragment thereof,
c. a functional variant of (b) having at least 80% sequence identity to SEQ ID NO. 1 or 2 or a functional fragment thereof,
d. a peptide of about 64 to about 100 amino acids in length, wherein the peptide comprises SEQ ID NO. 1 or 2,
e. a functional variant of (d), wherein the peptide comprises an amino acid sequence having at least 80% sequence identity to SEQ ID NO. 1 or 2,
f. a casein hydrolysate comprising one or more peptides consisting of SEQ ID NO. 1 or 2, wherein the hydrosylate comprises about 51 to about 100% by weight of the one or more peptides,
g. sweet whey, and
h. any combination of any two or more of (a) to (g).

48. A method of claim 47, comprising the administration of a composition comprising about 51 to about 100% by weight of the one or more agents as defined in claim 18.


SEQUENCE LISTING (4 sequences)

SEQ ID NO: 1 - protein, 64 aa, Bos taurus
  ref <308>: DATABASE ACCESSION NUMBER: NP_776719
  ref <309>: DATABASE ENTRY DATE: 2008-09-21
  ref <313>: RELEVANT RESIDUES IN SEQ ID NO: (127)..(190)
MAIPPKKNQDKTEIPTINTIASGEPTSTPTTEAVESTVATLEDSPEVIESPPEINTVQVTSTAV

SEQ ID NO: 2 - protein, 64 aa, Bos taurus
  ref <301>: AUTHORS: Brody E. P
  ref <302>: TITLE: Biological activities of bovine glycomacropeptide
  ref <303>: JOURNAL: British Journal of Nutrition
  ref <304>: VOLUME: 84
  ref <306>: PAGES: S39-46
  ref <307>: DATE: 2000
  ref <313>: RELEVANT RESIDUES IN SEQ ID NO: (1)..(64)
MAIPPKKNQDKTEIPTINTIASGEPTSTPTIEAVESTVATLEASPEVIESPPEINTVQVTSTAV

SEQ ID NO: 3 - protein, 169 aa, Bos taurus
  ref <308>: DATABASE ACCESSION NUMBER: NP_776719
  ref <309>: DATABASE ENTRY DATE: 2008-09-21
  ref <313>: RELEVANT RESIDUES IN SEQ ID NO: (22)..(190)
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 4 - protein, 105 aa, Bos taurus
  ref <308>: DATABASE ACCESSION NUMBER: NP_776719
  ref <309>: DATABASE ENTRY DATE: 2008-09-21
  ref <313>: RELEVANT RESIDUES IN SEQ ID NO: (22)..(126)
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSF